(19) United States
(12) Patent Application Publication
(10) Pub. No.: US 2009/0036985 A1
(43) Pub. Date: Feb. 5, 2009

(54) Vertebral Fusion Parts, Devices and Methods

(75) Inventor: Jeremy Whiting, West Yorkshire (GB)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DEPUY INTERNATIONAL LIMITED, Leeds (GB)

(21) Appl. No.: 10/586,843

(22) PCT Filed: Jan. 21, 2005
(86) PCT No.: PCT/GB2005/000165

§ 371 Date: Oct. 16, 2008

(30) Foreign Application Priority Data
Jan. 22, 2004 (GB) 0401362.9

(51) Int. Cl. A61F 2/44; A61B 19/00

(52) U.S. Cl. 623/17.11; 128/898

(57) ABSTRACT

End caps and end cap and end plate assemblies for use with a cage or mesh, for use in surgical vertebral fusion procedures are described. A first end cap comprises an annular body and a projection extending from an outer side wall of the body to limit the travel of the end cap into the cage. At least a first and a second flexible member extend from an under side of the body. Each flexible member has a grip on an outer facing surface to retain the end cap on the end of the cage. A second end cap comprises an annular body having an uppermost surface, a projection extending from an outer side wall of the body and a formation adapted to mate with a formation in an end plate, the end plate having a vertebra engaging surface, such that the outermost surface of the body is flush with the vertebra engaging surface of the end plate. When inserted in an end of the cage in use, the projection engages an edge of the cage in more than one location to limit the travel of the end cap into the cage.

The present invention relates to parts, devices and methods for use in fusing vertebrae, and in particular to end caps, end plates and end cap and end plate assemblies for use with a cage or mesh, and related methods, for use in surgical vertebral fusion procedures.

Vertebral fusion is a surgical technique used to treat back problems and conditions by fusing together adjacent vertebrae so as to prevent their relative movement. For example, it can be used when an intervertebral disc has ruptured. The technique aims to cause bone growth between adjacent vertebra. An implant is often used to maintain the spacing apart of the vertebra as corresponding to that when the intervertebral disc is still present.

A variety of techniques can be used and many techniques can require each using a different intervertebral fusion device which can comprise or require various components, implants and/or instruments. Often multiple parts need to be correctly positioned in situ in the intervertebral cavity which can be a task requiring considerable skill and experience on the part of the surgeon.

Therefore, there is a need for intervertebral fusion components which simplify and make easier the surgical procedure and help to increase its reliability.

According to a first aspect of the present invention, there is provided an end cap for an intervertebral cage for use in an intervertebral fusion procedure. The end cap can comprise an annular body. The end cap can include a projection extending from an outer side wall of the body. When inserted in an end of the cage in use, the projection engages an edge of the cage to limit the travel of the end cap into the cage. The end cap can have a first and a second flexible member. Each flexible member can have a grip on an outer facing surface. The grips engage an inner surface of the cage to retain the end cap on the end of the cage.

The projection helps to ensure that the end cap is automatically correctly seated in the cage by the surgeon in use as the surgeon can use feel. The end cap can be securely retained in cage and hence the surgeon can more easily handle and manipulate the cage and end cap assembly, without having to worry about the end cap falling out.

The first flexible member and the second flexible member can be disposed on substantially opposite sides of the annular body. This helps to ensure that the cap is securely held on the cage.

The end cap can have more than two flexible members. The end cap can include a third flexible member extending from an under side of the body and also having a grip on an outer facing surface. The use of three flexible members helps to ensure that the end cap is securely held on the cage by providing three points of contact around the cage.

The flexible members can be equi-angularly disposed around the annular body. Each neighboring pair of flexible members can be separated by the same angle. For example, for two members they are separated by 180°, for three members neighboring members are separated by 120°, etc.

The grip can be provided by any surface formation. The grip can be provided by at least one barbed or tooth like formation. More than one barbed or tooth like formation can be used as the grip. A barb or tooth helps to retain the cap in a cage as the edge of the barb or tooth can engage with the apertures in the cage body. Alternatively, a roughened surface can provide a grip or a different material having a high co-efficient of friction can be used to provide the grip.

The projection can have a plurality of parts for engaging the cage edge at different positions. The projection can extend around the entire periphery of the annular body. The projection can extend from at least three separate positions around the periphery of the annular body.

The end cap can include a formation adapted for mating with a formation in an end plate having a vertebra engaging surface presented in use to a vertebral surface, and wherein the end cap has an outermost surface, such that when the end cap formation mates with the end plate formation, the vertebra engaging surface and outermost surface are substantially flush. The end cap formation can be a rim or wall part of the body part. The formation can extend partially or wholly around the end cap. The formation can have a single part or multiple parts.

The end cap formation can be adapted to mate with the end plate by presenting the end cap to an under side of the end plate. In this way the end cap can be engaged with the end plate in situ with the end plate already mounted in a vertebra.

According to a second aspect of the invention, there is provided a kit of parts for assembling into an intervertebral fusion device assembly. The kit of parts can comprise an end cap as mentioned above and an end plate. The end plate can have a vertebra engaging surface presented in use to a vertebral surface and a formation adapted to mate with a formation of the end cap. When assembled, the vertebra engaging surface of the end plate and the outermost surface of the end cap are substantially flush. The kit can also include a cage. The kit can also include a further end cap and/or end plate.

According to a third aspect of the invention, a method for use in an intervertebral fusion surgical procedure is provided. The method can comprise push fitting a first annular end cap into a first end of a cage by deforming at least two flexible members of the first end cap. The interior of the cage can be packed with a material which enhances bone fusion via an opening of the end cap. As the end cap is securely retained in place, it is easier for the surgeon to pack the cage without having to also control or manipulate the end cap.

The method can further comprise push fitting a second annular end cap into a second end of a cage by deforming at least two flexible members of the second end cap prior to packing the cage. This helps to ensure that the end caps are both securely attached to the cage and means that attaching the end caps does not disturb the material packed in the cage.

The method can further comprise mounting an end plate onto a surface of a vertebra in an intervertebral space separately to an end cap and subsequently introducing the packed cage into the intervertebral space and engaging the end cap with the end plate. This simplifies the surgical procedure as the end plate can be inserted into the intervertebral space separately from the end cap and cage.

Engaging the end cap with the end plate can present a substantially flush surface to the surface of the vertebra which exposes the material to the surface of the vertebra. This helps to facilitate bone growth and hence vertebral fusion by increasing the amount of material that is presented to the exposed surface of the vertebra.

According to a fourth aspect of the invention, an end cap for an intervertebral cage for use in an intervertebral fusion procedure is provided. The end cap can comprise an annular body having an outermost surface. The end cap can have a formation adapted to mate with a formation in an end plate. The end plate has a vertebra engaging surface, and the outermost surface of the body is flush with the vertebra engaging surface of the end plate in use. The end cap can also have a projection extending from the body. The projection can engages an edge of the cage in more than one location to limit the travel of the end cap into the cage.

Providing an end cap which can be used with an end plate to ensure that the contents of the cage are more reliably exposed to a vertebra helps to facilitate bone growth and hence vertebral fusion by increasing the amount of material that can presented to the exposed surface of the vertebra.

The end cap formation can be a wall, rim or edge of the annular body part. The wall, rim or edge can be continuous and extend around the entire end cap, or can be composed of a plurality of separate parts. At least three parts can be provided.

The end cap formation can be adapted to engage with an under side of the end plate. This allows the end cap to be engaged with an end plate in situ with the end plate already mounted in a vertebra.

The end cap formation can comprise a flange. The flange can extend around the entire periphery of the end cap. The formation can includes at least three separate elements disposed around the end cap. The formation can be provided as a part of the projection. That is the element of the end cap providing the projection also, at least partially, provides the end cap formation which mates with the end pate in use. The formation can be provided by a shoulder of the projection.

The end cap can further comprise at least a first and a second flexible member extending from an under side of the body. Each flexible member can have a grip on an outer facing surface. When inserted in an end of the cage in use, the grips can engage an inner surface of the cage to retain the end cap on the end of the cage. The flexible members can also be resilient. Other preferred features of the flexible members are mentioned above in connection with the first aspect of the invention.

According to a fifth aspect of the invention, there is provided a method for use in an intervertebral fusion surgical procedure. The method can comprise mounting an end plate onto a surface of a vertebra in an intervertebral space separately to an end cap. The method can further comprise connecting an end cap to an end of a cage. The interior of the cage can be packed with a material which enhances bone fusion. The packed cage can be introduced into the intervertebral space and the end cap engaged with the end plate. An outermost most surface of the end cap can be flush with the vertebra engaging surface of the end plate. This helps to present the material to the vertebral surface thereby more reliably facilitating bone growth and vertebral fusion.

The method can further comprise mounting a further end plate onto a surface of a further vertebra in the intervertebral space separately to any end caps, the end plate having a further vertebra engaging surface, connecting a second end cap to a second end of the cage, the end cap having an outermost surface and a formation adapted to mate with a formation in the further end plate, and wherein introducing the packed cage into the intervertebral space further comprises engaging the second end cap with the further end plate such that the outermost surface of the further end cap is flush with the further vertebra engaging surface of the further end plate to present the material to the surface of the further vertebra. This simplifies the surgical procedure and reduces the size of the assembly that need to be inserted via an incision as the end plates can be inserted separately to the cage and end caps.

The method can further comprise connecting the first and/or second end caps by push fitting the or each end cap into an end of a cage by deforming at least two flexible members extending from an under side of the end cap prior to packing the cage. Connecting the end caps to the cage before packing the cage ensures that connecting the end caps does not disturb the material packed in the cage.

According to a sixth aspect of the invention, there is provided an intervertebral fusion assembly, comprising a cage, an annular end cap mounted on an end of the cage and having a plurality of resilient flexible limbs which grip an liner surface of the cage, and an end plate having an aperture therein and a surface for engaging a vertebral surface. In use the end plate can be mounted on the end cap by mating the end cap with the aperture and with the surface for engaging a vertebral surface being flush with an outermost surface of the end cap.

An embodiment of the invention will now be described, by way of example only, and with reference to the accompanying drawings, in which:

FIG. 1 shows a perspective view of an end cap according to the invention;

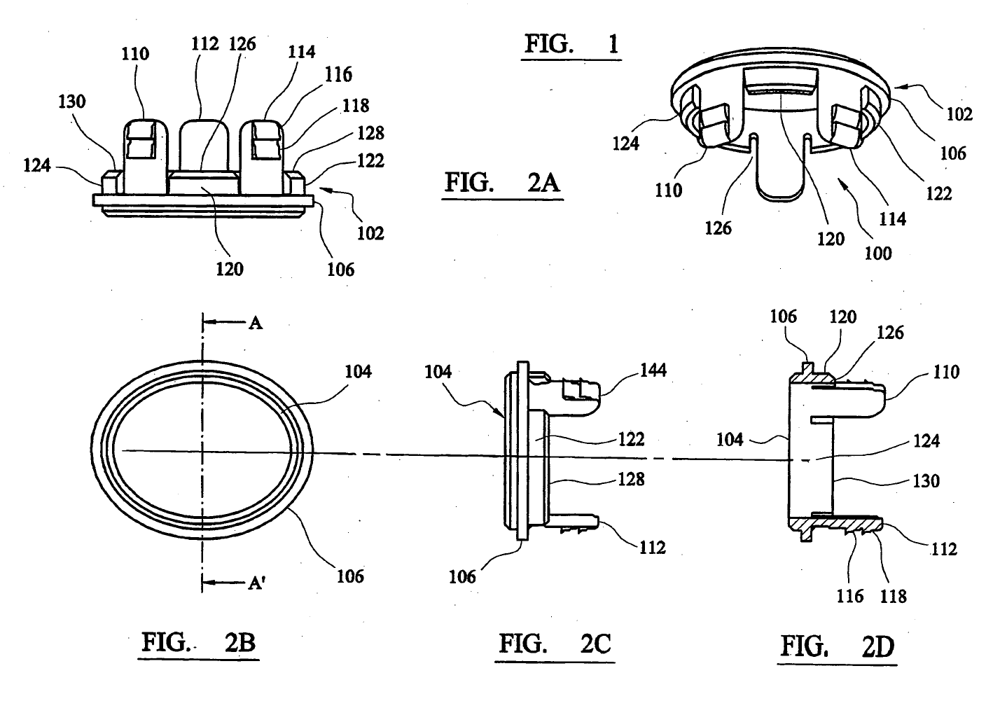

FIGS. 2A, 2B, 2C and 2D respectively show a side, plan, end and cross sectional view along line AA′ of the end cap of FIG. 1;

FIG. 3 shows a perspective view of an end plate part of the invention;

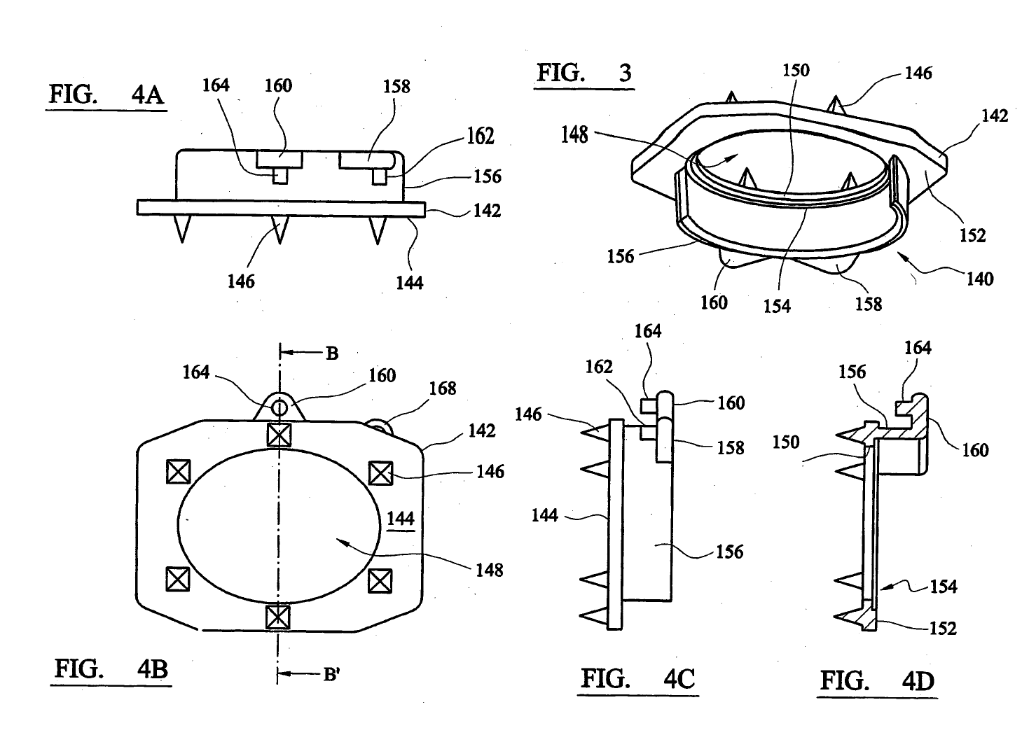

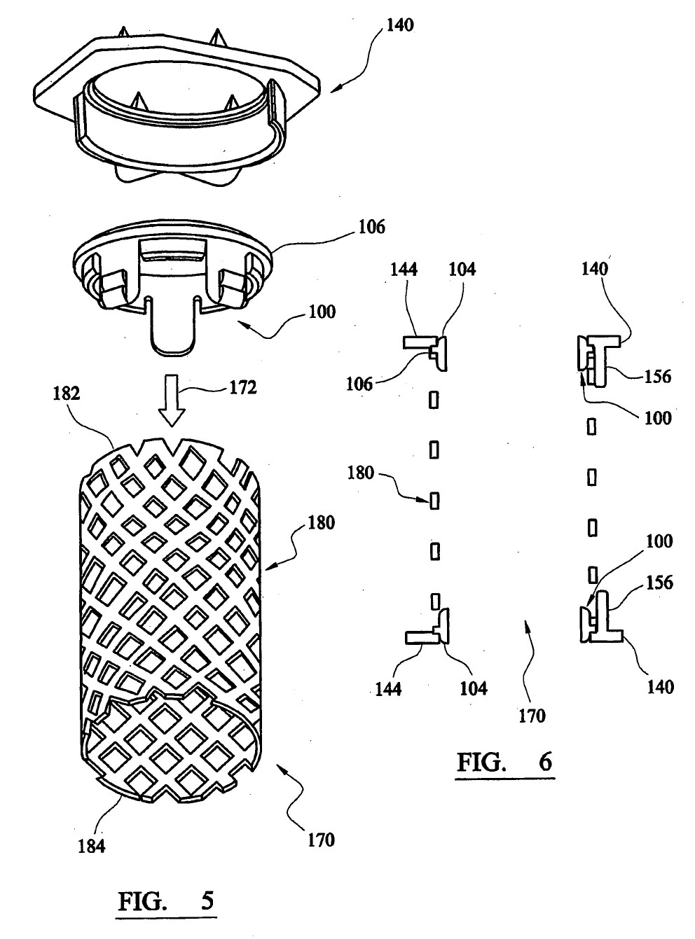

FIGS. 4A, 4B, 4C and 4D respectively show a side, plan, end and cross sectional view along line BB′ of the end plate of FIG. 3;

FIG. 5 shows a perspective view of an exploded assembly of the end cap of FIG. 1, the end plate of FIG. 3 and a cage part;

FIG. 6 shows a cross sectional view of an intervertebral fusion device assembly including end caps as shown in FIG. 1, end plates as shown in FIG. 3 and a cage part; and

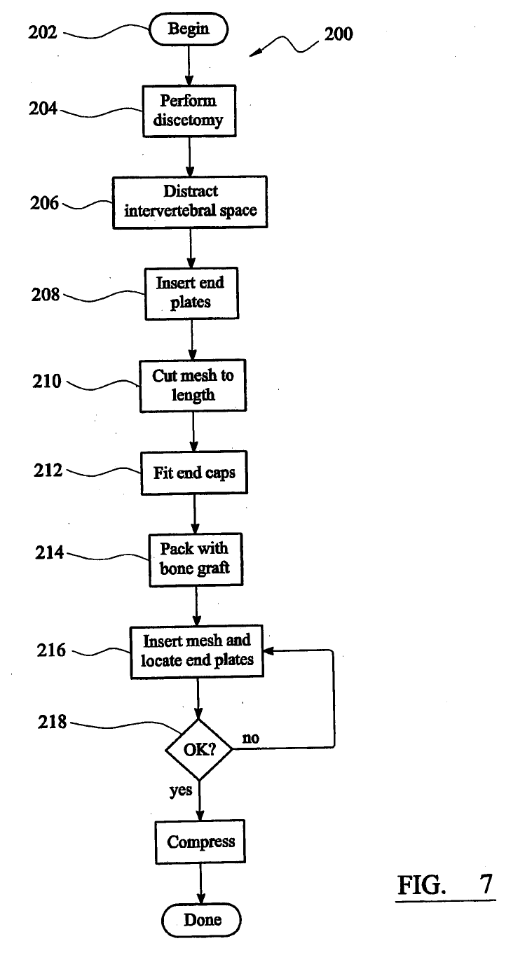

FIG. 7 shows a flow chart illustrating a method of using the intervertebral fusion device assembly during a surgical vertebrae fusion procedure.

Similar items in different Figures share common reference numerals unless indicated otherwise.

With reference to FIGS. 1 and 2A to 2D there is shown an end cap 100 according to the present invention. FIG. 2D shows a cross section along line AA′ of FIG. 2B. The end cap 100 has a generally annular body part 102 having an oval shape. In alternative embodiments, the end cap can have other regular and irregular, polygonal and curved shapes, including circular and ‘kidney’ shapes. Toward an exterior end of the end cap, the body has an outermost surface 104. An outer peripheral edge of the outermost surface 104 of body 102 is chamfered around its periphery.

Body 102 has a flange 106 projecting from a side wall and extending around the entire periphery of the end cap 100. In alternate embodiments, this projecting formation can be comprised of at least two or more separate projections, such as lugs, with gaps between them. An inner end of end cap 100 has first 110, second 112 and third 114 flexible tongues depending from the body 102. Each flexible tongue 110, 112, 114 has a grip in the form of two barbs or teeth 116, 118 on an outer surface thereof. Flexible tongues 110, 112 and 114 provide flexible members, or limbs, and are equi-angularly disposed about the end cap body, i.e. there is an angle of substantially 120′ between neighbouring pairs of flexible tongues. Flexible tongues are also resilient. The provision of three flexible tongues, which in use grip against an inner surface of a cage as will be described in greater detail below, helps to ensure correct location of the end cap in the cage and its secure retention therein. More or fewer than three flexible tongues can be provided. When two tongues are provided, it is preferred that they are located on opposed sides of the annular body, i.e. 180° apart.

Annular body 102 also includes first, second and third skirt parts 120, 122, 124 each disposed between a neighbouring pair of flexible tongues and with a gap between the end of each skirt part and the adjacent tongue, e.g. gap 126. Each skirt 120, 122, 124 has an edge providing an inner most surface 126, 128, 130 of the end cap and which edge is chamfered along its length.

The end cap can be made of any biocompatible material, and is preferably made of surgical grade titanium, although it can also be made of stainless steel or non-metallic materials. The end cap has a unitary construction, i.e., it has a single part. The end cap can be manufactured by machining a single piece of material.

With reference to FIGS. 3 and 4A to 4D there is shown an embodiment of an end plate 140 particularly suitable for use with end cap 100 of the present invention. FIG. 4D shows a cross section along line BB′ of FIG. 4B. End plate 140 includes a plate 142 with a vertebral surface engaging surface 144 bearing six spikes 146 for penetrating a vertebra and securing the end plate to the vertebra. Plate 142 has an oval aperture 148 therein defined by an inner edge 150 of plate 142. An inner part of aperture defining edge 150 is chamfered around its entire periphery.

An under or inner side of the plate 152, opposed to the vertebra engaging side 144, has a recessed portion 154 extending around the periphery of aperture 148. A wall 156 extends away from the under surface 152 of plate 142 and approximately halfway around the aperture 148. The free edge of wall 156 bears a chamfer along its length. A first lug 158 and a second lug 160 provide projections extending outwardly from an outer surface of wall 156. Each of lugs 158 and 160 bear a stud or other connecting mechanism by which an instrument can be releasably attached to the end plate for insertion in an intervertebral cavity.

End plate 140 has a unitary construction and is made of surgical grade titanium. However, the end plate can be made of any biocompatible material, such as stainless steel or non-metallic materials. The end plate can be manufactured by machining a single piece of material.

End plate 140 is generally configured, shaped and sized to co-operate with end cap 100 as will now be described with particular reference to FIG. 5.

FIG. 5 shows a perspective view of the components of an intervertebral fusion device or assembly 170 including end plate 140, end cap 100 and cage or mesh element 180. Cage 180 is generally cylindrical having an oval cross-section and is made of a titanium mesh. Cage 180 can also be made of other biocompatible materials, such as stainless steel or non-metallic materials. Cage 180 has a first edge 182 and a second edge 184 at opposed ends of the cage. Edges 182, 184 are defined by mesh parts and also recess parts formed by incomplete apertures of the mesh. As illustrated in FIG. 5, cage 180, end cap 100 and end plate 140 are all in registration centred on the central longitudinal axis of cage 180 passing through the centre of the oval.

To assemble the intervertebral fusion device, end cap 100 is presented to the open end of cage 180 and push fitted into the open end of cage 170 in the direction of arrow 172. As tongues 110, 112 and 114 are flexible, they deform sufficiently to allow end cap 100 to be introduced into the free end of cage 170 until flange 106 engages with the outer edge 182 of cage 170 to limit further motion of end cap 100 into cage 180. The chamfered edges of skirts 120, 122, 124 also help to facilitate engagement of the end cap and cage and the skirts also help to centre and securely seat the end cap in the end of cage 170. Tongues 110, 112 and 114 are also resilient members and urge their barbs into engagement with the inner surface of cage 180 so as to retain end cap 100 in cage 180. The outer side of end cap 100 can then be presented to the under side of end plate 140 by engaging the outer part of annular body 102 into aperture 148 of end plate 140.

FIG. 6 shows a schematic cross-sectional diagram through an assembled intervertebral fusion device 170 including cage 180 having an end cap in each end and an end plate mounted on each end cap. As can be seen in FIG. 6, the flange 106 of each end cap 100 engages against and around the edges 182, 184 of the cage to limit the motion of the end cap into the cage. As the flange engages around the entire edge of the cage, this helps to ensure that the end cap is correctly seated. In other embodiments in which the flange is made of a number of separate parts, each parts should be sufficiently long to span any recesses in edges 182, 184 to ensure correct seating of the end cap. Further, end plate 140 is supported by an upper surface of flange 106 engaging in recess 154. The outer part of annular body 104 is retained within the inner aperture defining edge 150 of end plate 140 and the various chamfers on the edges of the various parts facilitate their engagement.

The parts of the end plate and end cap are configured and sized such that when assembled, the outermost surface 104 of end cap 100 is substantially flush with the vertebra engaging surface 144 of end plate 140. In use this facilitates contact between bone growth medium packed within cage 180 and an exposed vertebra surface so as to promote bone fusion as will be described in greater detail below.

FIG. 7 shows a flowchart illustrating some of the steps of a intervertebral fusion procedure 200 using the intervertebral fusion device assembly 170 illustrated in FIGS. 5 and 6. It will be appreciated that there are other surgical actions carried out before and after the surgical operations illustrated in FIG. 7 but these have been omitted so as not to obscure the nature of the present invention.

The surgical procedure is begun at step 202 and at step 204 a discectomy is performed to remove a damaged disc from the intervertebral space. At step 206 an instrument is inserted between adjacent vertebrae and the intervertebral space is distracted to provide space for inserting and accommodating the parts of the intervertebral fusion device. At step 208, an instrument is used to insert a first end plate into the intervertebral space. The end plate is positioned and then urged into engagement with the surface of one of the vertebra and secured in place by the action of spikes 146 in the surface of the vertebra. A second end plate is also inserted into the intervertebral space, aligned with the first end plate and engaged with the surface of the other vertebra.

At step 210, the surgeon determines an appropriate length of cage and cuts a length of titanium mesh to provide an appropriate length cage 180. Step 210 can be optional as a standard length cage can be used in which case step 210 can be omitted.

At step 212, an end cap is push fitted into each end of the cage. The end caps are retained therein by the resilient tongues gripping the inner surface of the cage and the correct extent of insertion of the end caps into the cage is ensured by flange 106 abutting the end of the cage. At step 214, the surgeon packs the cage with bone graft, or any other medium suitable for enhancing the growth of bone between the vertebra to cause them to fuse. The cage is packed via the aperture of the upper most of the end caps and the cage is preferably overfilled so that the bone graft material stands proud of the outermost surface 104 of end cap 100.

At step 216, the cage and end cap assembly is inserted into the intervertebral space and the lower end cap of the assembly is inserted into the aperture 148 of the lower end plate and guided into the correct position by wall 156 and into correct engagement with the end plate by the co-operation of the annular body of the end cap mating with aperture 148. The upper end cap should be substantially aligned with the upper end plate and is guided into the correct position by wall 156 such that the centre of the end cap is substantially aligned with the centre of aperture 148. At step 218, the surgeon can determine whether they are satisfied with the alignment of the end plates and end caps and if not some adjustment of the positions of the end caps and end plates can be carried out. When the surgeon is satisfied with the alignment of the cage, end caps and end plates, then at step 220 the distraction instrument is released allowing the vertebra to compress the assembly between them.

As the vertebra engaging surface of each of the end plates are substantially flush with the outermost surfaces of the end caps, the material packed into the cage is more effectively presented to the surfaces of the vertebra thereby promoting bone growth between the vertebra and improving the reliability of the procedure and the resulting fusion of the vertebrae.

As will be appreciated, a number of the features of the end plates, end cap and assembly can be changed and/or modified and/or omitted. Similarly a number of the steps in the method described above can be optional and/or their sequence changed as will be apparent to a person of ordinary skill in the art.

For example, it is not essential to locate the end plates in the intervertebral space before inserting the mesh and end cap assembly. Instead, the end plates can be assembled on to the mesh and end cap assembly and the entire assembly inserted into the intervertebral space. It can be preferred not to have spike formations 146 on the vertebra engaging surface of the end plate for this procedure.

Further, more than one assembly can be inserted into the same intervertebral space. Furthermore, although the assembly is shown as having a substantially oval cross-sectional shape, other curved cross-sectional shapes, such as circular, kidney shaped, and more complex cross-sectional shapes can be used. Further, the invention is not limited to curved shapes and polygonal shapes, including irregular and regular polygons can be used instead.

1. An end cap for an intervertebral cage for use in an intervertebral fusion procedure, the end cap comprising:
an annular body having an outermost surface;
a projection extending from an outer side wall of the body; and
a formation adapted to mate with a formation in an end plate, the end plate having a vertebra engaging surface, such that the outermost surface of the body is flush with the vertebra engaging surface of the end plate and wherein, when inserted in an end of the cage in use, the projection engages an edge of the cage in more than one location to limit the travel of the end cap into the cage.

2. An end cap as claimed in claim 1, wherein the end cap formation is adapted to engage with an under side of the end plate.

3. An end cap as claimed in claim 1, wherein the end cap formation comprises a flange.

4. An end cap as claimed in claim 3, wherein the flange extends around the entire periphery of the end cap.

5. An end cap as claimed in claim 3, wherein the formation includes at least three separate elements disposed around the end cap.

6. An end cap as claimed in claim 5, wherein the formation is provided as a part of the projection.

7. An end cap as claimed in claim 6, wherein the formation is provided by a shoulder of the projection.

8. An end cap as claimed in claim 7, and further comprising at least a first and a second flexible member extending from an under side of the body, each flexible member having a grip on an outer facing surface wherein, when inserted in an end of the cage in use, the grips engage an inner surface of the cage to retain the end cap on the end of the cage.

9. An end cap as claimed in claim 8, wherein the first flexible member and the second flexible member are disposed on substantially opposite sides of the annular body.

10. An end cap as claimed in claim 8, wherein the end cap includes a third flexible member extending from an under side of the body and having a grip on an outer facing surface.

11. An end cap as claimed in claim 8, wherein each neighboring pair of flexible members is separated by the same angle.

12. An end cap as claimed in claim 8, wherein the grip is provided by at least one barbed formation.

13. An end cap for an intervertebral cage for use in an intervertebral fusion procedure, the end cap comprising:
an annular body;
a projection extending from an outer side wall of the body; and
at least a first and a second flexible member extending from an under side of the body, each flexible member having a grip on an outer facing surface wherein, when inserted in an end of the cage in use, the projection engages an edge of the cage in more than one location to limit the travel of the end cap into the cage and the grips engage an inner surface of the cage to retain the end cap on the end of the cage.

14. An end cap as claimed in claim 13, wherein the first flexible member and the second flexible member are disposed on substantially opposite sides of the annular body.

15. An end cap as claimed in claim 13, wherein the end cap includes a third flexible member extending from an under side of the body and having a grip on an outer facing surface.

16. An end cap as claimed in claim 13, wherein each neighbouring pair of flexible members is separated by the same angle.

17. An end cap as claimed in claim 13, wherein the grip is provided by at least one barbed formation.

18. An end cap as claimed in claim 13, wherein the projection extends around the entire periphery of the annular body.

19. An end cap as claimed in claim 13, wherein the projection extends from at least three separate positions around the periphery of the annular body.

20. An end cap as claimed in claim 13 and including a formation adapted for mating with a formation in an end plate having a vertebra engaging surface presented in use to a vertebral surface, and wherein the end cap has an outermost surface, such that when the end cap formation mates with the end plate formation, the vertebra engaging surface and outermost surface are substantially flush.

21. An end cap as claimed in claim 20, wherein the end cap formation is adapted to mates with the end plate by presenting the end cap to an under side of the end plate.

22. A kit of parts for assembling into an intervertebral fusion device assembly, the kit of parts comprising:
an end cap comprising
an annular body,
a projection extending from an outer side wall of the body, and
at least a first and a second flexible member extending from an under side of the body, each flexible member having a grip on an outer facing surface wherein, when inserted in an end of the cage in use, the projection engages an edge of the cage in more than one location to limit the travel of the end cap into the cage and the grips engage an inner surface of the cage to retain the end cap on the end of the cage; and
an end plate having a vertebra engaging surface presented in use to a vertebral surface and a formation adapted to mate with the formation of the end cap such that when assembled, the vertebra engaging surface of the end plate and the outermost surface of the end cap are substantially flush.

23. A kit of parts as claimed in claim 22, and further comprising a cage.

24. A method for use in an intervertebral fusion surgical procedure, comprising:
push fitting a first annular end cap into a first end of a cage by deforming at least two flexible members extending from an under side of the first end cap; and
packing the interior of the cage with a material which enhances bone fusion via an opening of the end cap.

25. A method as claimed in claim 24, and further comprising:
push fitting a second annular end cap into a second end of a cage by deforming at least two flexible members extending from an under side of the second end cap prior to packing the cage.

26. A method as claimed in claim 14, further comprising:
mounting an end plate onto a surface of a vertebra in an intervertebral space separately to an end cap; and
subsequently introducing the packed cage into the intervertebral space and engaging the end cap with the end plate.

27. A method as claimed in claim 26, wherein engaging the end cap with the end plate presents a substantially flush surface to the surface of the vertebra which exposes the material to the surface of the vertebra.

28. A method for use in an intervertebral fusion surgical procedure, comprising:
mounting an end plate onto a surface of a vertebra in an intervertebral space separately to an end cap, the end plate having a vertebra engaging surface;
connecting a first end cap to a first end of a cage, the end cap having an outermost surface and a formation adapted to mate with a formation in the end plate;
packing the interior of the cage with a material which enhances bone fusion; and
introducing the packed cage into the intervertebral space and engaging the end cap with the end plate such that the outermost most surface of the end cap is flush with the vertebra engaging surface of the end plate to present the material to the vertebral surface.

29. The method as claimed in claim 28, further comprising:
mounting a further end plate onto a surface of a further vertebra in the intervertebral space separately to any end caps, the end plate having a further vertebra engaging surface;
connecting a second end cap to a second end of the cage, the end cap having an outermost surface and a formation adapted to mate with a formation in the further end plate, and
wherein introducing the packed cage into the intervertebral space further comprises engaging the second end cap with the further end plate such that the outermost surface of the further end cap is flush with the further vertebra engaging surface of the further end plate to present the material to the surface of the further vertebra.

30. The method of claim 28, wherein connecting the first and/or second end cap comprises push fitting the or each end cap into an end of a cage by deforming at least two flexible members extending from an under side of the end cap prior to packing the cage.

31. An intervertebral fusion assembly, comprising:
a cage having a first end and an inner surface;
an annular end cap mounted on the first end of the cage and having an outermost surface and three resilient flexible limbs which grip the inner surface of the cage; and
an end plate having an aperture therein and a surface for engaging a vertebral surface in use wherein the end plate is mounted on the end cap by mating the end cap with the aperture and with the surface for engaging a vertebral surface being flush with the outermost surface of the end cap.
